SEQUENCE LISTING (12 sequences)

SEQ ID NO: 1 - DNA, 392 bp, Hordeum vulgare
  CDS (1)..(390)
gaaaagcatatgattgaaggtcgtatgaaaagctgctgccgtagcaccctgggtcgtaactgctataacctgtgccgtgttcgtggtgcgcagaaactgtgcgcgggtgtttgccgttgcaaactgaccagcagcggtaaatgcccgaccggttttccgaaaatgattgaaggtcgtacgctggcgctggttagcaacagcgatgaaccggataccgttaaatattgcaacctgggttgccgtgcgagcatgtgcgattatatggttaacgcggcggcggatgatgaagaaatgaaactgtatctggaaaactgcggtgatgcgtgcgttaacttttgcaacggtgatgcgggtctgaccagcctgaccgcgtgataggatccggctgctaa

SEQ ID NO: 2 - protein, 124 aa, Hordeum vulgare
EKHMIEGRMKSCCRSTLGRNCYNLCRVRGAQKLCAGVCRCKLTSSGKCPTGFPKMIEGRTLALVSNSDEPDTVKYCNLGCRASMCDYMVNAAADDEEMKLYLENCGDACVNFCNGDAGLTSLTA

SEQ ID NO: 3 - protein, 4 aa, Hordeum vulgare
DPAA

SEQ ID NO: 4 - DNA, 1649 bp, Hordeum vulgare
  CDS (1)..(1647)
gacaagcatatgattgaaggtcgtaaaagctgctgccgtagcaccctgggtcgtaactgctataacctgtgccgtgttcgtggtgcgcagaaactgtgcgcgggtgtttgccgttgcaaactgaccagcagcggtaaatgcccgaccggttttccgaaaatgattgaaggtcgttcggatgttgtccagctgaagaaggacaccttcgacgacttcatcaagacgaatgaccttgttctcgccgaattcttcgcgccgtggtgcggtcactgcaaggctctcgcccccgagtacgaggaggctgcgaccacactgaaggagaagaacatcaagctcgccaaggtggactgcacagaggagacggacctctgccaacaacatggtgttgagggctacccgactctcaaggtcttccgcggccttgacaacgtctccccctacaagggccagcgcaaggctgctgctatcacctcgtacatgatcaagcagtctctgcccgccgtgtccgaggtcacgaaggacaacctggaggagttcaagaaggccgacaaggccgtccttgtcgcctatgtggatgcttccgacaaggcgtccagtgaggttttcacccaggtcgccgagaagctgcgcgacaactacccgttcggctccagcagcgatgctgcgctggccgaggctgagggcgtcaaggctcccgctatcgtcctttacaaggactttgatgagggcaaggcggtcttctccgagaagttcgaggtggaggcgatcgagaagttcgccaagacgggcgccaccccgctcattggcgagattggccccgaaacctactccgactacatgtcggccggcatccctctggcctacattttcgccgaaacggccgaggagcggaaggagctcagcgacaagctcaagccgatcgccgaggctcagcgcggcgtcattaactttggtactattgacgccaaggcttttggtgcccacgccggcaacctgaacctgaagaccgacaagttccccgccttcgccatccaggaggtcgccaagaaccagaagttccccttcgatcaggagaaggagatcaccttcgaggcgatcaaggctttcgtcgacgactttgtcgccggtaagatcgagcccagcatcaagtcggagccgatccctgagaagcaggagggccccgtcaccgtcgtcgttgccaagaactacaatgagatcgtcctggacgacaccaaggatgtgctgattgagttctacgccccgtggtgcggccactgcaaggccctggctcccaagtacgaggagctcggcgccctgtatgccaagagcgagttcaaggaccgggtcgtcatcgccaaggttgatgccacggccaacgacgttcccgatgagatccagggattccccaccatcaagctgtacccggccggtgccaagggtcagcccgtcacctactctggctcgcgcactgtcgaggacctcatcaagttcatcgccgagaacggcaagtacaaggccgccatctcggaggatgccgaggagacgtcgtccgcaaccgagacgaccaccgagacggccaccaagtcggaggaggctgccaaggagacggcgacggagcacgacgagctctgataggatccggctgctaa

SEQ ID NO: 5 - protein, 543 aa, Hordeum vulgare
DKHMIEGRKSCCRSTLGRNCYNLCRVRGAQKLCAGVCRCKLTSSGKCPTGFPKMIEGRSDVVQLKKDTFDDFIKTNDLVLAEFFAPWCGHCKALAPEYEEAATTLKEKNIKLAKVDCTEETDLCQQHGVEGYPTLKVFRGLDNVSPYKGQRKAAAITSYMIKQSLPAVSEVTKDNLEEFKKADKAVLVAYVDASDKASSEVFTQVAEKLRDNYPFGSSSDAALAEAEGVKAPAIVLYKDFDEGKAVFSEKFEVEAIEKFAKTGATPLIGEIGPETYSDYMSAGIPLAYIFAETAEERKELSDKLKPIAEAQRGVINFGTIDAKAFGAHAGNLNLKTDKFPAFAIQEVAKNQKFPFDQEKEITFEAIKAFVDDFVAGKIEPSIKSEPIPEKQEGPVTVVVAKNYNEIVLDDTKDVLIEFYAPWCGHCKALAPKYEELGALYAKSEFKDRVVIAKVDATANDVPDEIQGFPTIKLYPAGAKGQPVTYSGSRTVEDLIKFIAENGKYKAAISEDAEETSSATETTTETATKSEEAAKETATEHDEL

SEQ ID NO: 6 - protein, 4 aa, Hordeum vulgare
DPAA

SEQ ID NO: 7 - DNA, 182 bp, Hordeum vulgare
  CDS (1)..(180)
gacaagcatatgattgaaggtcgtatgaaaagctgctgccgtagcaccctgggtcgtaactgctataacctgtgccgtgttcgtggtgcgcagaaactgtgcgcgggtgtttgccgttgcaaactgaccagcagcggtaaatgcccgaccggttttccgaaatgataggatccggctgctaa

SEQ ID NO: 8 - protein, 54 aa, Hordeum vulgare
DKHMIEGRMKSCCRSTLGRNCYNLCRVRGAQKLCAGVCRCKLTSSGKCPTGFPK

SEQ ID NO: 9 - protein, 4 aa, Hordeum vulgare
DPAA

SEQ ID NO: 10 - DNA, 284 bp, Hordeum vulgare
  CDS (1)..(282)
gacaagcatatgattgaaggtcgtaaaagctgctgccgtagcaccctgggtcgtaactgctataacctgtgccgtgttcgtggtgcgcagaaactgtgcgcgggtgtttgccgttgcaaactgaccagcagcggtaaatgcccgaccggttttccgaaaatgattgaaggtcgtgagacgtcgtccgcaaccgagacgaccaccgagacggccaccaagtcggaggaggctgccaaggagacggcgacggagcacgacgagctctgataggatccggctgctaa

SEQ ID NO: 11 - protein, 88 aa, Hordeum vulgare
DKHMIEGRKSCCRSTLGRNCYNLCRVRGAQKLCAGVCRCKLTSSGKCPTGFPKMIEGRETSSATETTTETATKSEEAAKETATEHDEL

SEQ ID NO: 12 - protein, 4 aa, Hordeum vulgare
DPAA

(19) United States
(12) Patent Application Publication
(10) Pub. No.: US 2003/0228654 A1
(43) Pub. Date: Dec. 11, 2003

(54) Method for producing antimicrobial protein and fusion protein

(75) Inventors: Takao Imaeda, Aichi-ken (JP); Yukio Yamada, Aichi-ken (JP); Masana Hirai, Aichi-ken (JP); Takashi Shimamura, Aichi-ken (JP); Katsunori Kohda, Aichi-ken (JP); Nobuhiko Muramoto, Aichi-ken (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOYOTA CHUO KENKYUSHO, Aichi-ken (JP)

(21) Appl. No.: 09/864,169

(22) Filed: May 25, 2001

(30) Foreign Application Priority Data
May 26, 2000 (JP) 2000-161090

(51) Int. Cl. C07K 14/47; C12P 21/02; C12N 1/21; C12N 15/74; C07H 21/04

(52) U.S. Cl. 435/69.1; 435/252.3; 435/320.1; 435/325; 530/350; 536/23.5

(57) ABSTRACT

A basic antimicrobial protein is activated by a partner protein having an isoelectric point below pH 7 and a chaperon function, by expressing an antimicrobially inactive fusion protein between the basic antimicrobial protein and the partner protein, recovering the fusion protein and separating the two proteins from each other. In such manner, an advantageous mass expression system of the basic antimicrobial protein having an appropriate disulfide bond as an active type is realized at lost cost.

### BACKGROUND OF THE INVENTION

1. Field of the Invention

The present invention relates to a mass expression system of a basic antimicrobial protein having a predetermined mode of an intramolecular disulfide bond as an active type.

2. Description of the Related Art

Via the application of genetic recombinant technology, proteins with various functions have increasingly been expressed in diversified expression systems such as bacteria, fungi, and mammalian animals. From the respect of efficient mass production of protein, expression systems of prokaryotic cells such as Escherichia coli as hosts are advantageous. On the other hand, the expression of antimicrobial protein, which draws attention in numerous industrial fields, presents a specific problem of toxicity to host cells. Another problem of antimicrobial protein with relatively low molecular weight is its decomposition in host cells. Therefore, the following techniques have been proposed for the production of antimicrobial protein in Escherichia coli, for example.

In one of the proposed methods, a fusion unit between antimicrobial protein and acid protein is formed into multimer, thereby masking the toxicity of the antimicrobial protein and simultaneously suppressing the decomposition thereof in cells [Jae H. Lee, II Minn, Chan B, et al. (1998) Protein Expression and Purification 12: 53-60]. In another method to gain the same effect as described above, fusion of antimicrobial protein with prochymosin to be expressed in an insoluble fraction is proposed [Chris Haught, Gregory D., Rajesh Subramanian, et al. (1998) Biotechnology and Bioengineering 57, 1: 55-61]. In still another method, fusion of antimicrobial protein with glutathione-S-transferase 3 (GST) is proposed [Kirill A. Martemyanov, Alexander S. Spirin, Anatory T. Gudkov (1996) Biotechnology Letters 18, 12: 1357-1362]. In a further method, fusion of antimicrobial protein with cellulose-binding domain (CBD) is proposed [Kevin L. Piers, Melissa H. Brown, et al. (1993) Gene 134, 1:7-13]. In a still further method, fusion of antimicrobial protein with protein A is proposed [L. Zhang, T. Falla, M. Wu, et al. (1998) Biochemical and Biophysical Res. Com. 247: 674-680].

Among antimicrobial proteins, some important ones are basic antimicrobial proteins, having cysteine on the amino acid sequences thereof. The active type thereof should have intramolecular disulfide bond of a predetermined mode. Representative examples of such basic antimicrobial protein include: thionin, PR protein, lipid transfer protein, and ribosome-inactivating protein, all from paints; difensin from plants, insects or humans; and the like.

These types of basic antimicrobial proteins have problems of toxicity to host cells and decomposition in host cells as described above. In addition, it is very difficult to recover basic antimicrobial proteins of active types having intramolecular disulfide bonds of an accurate predetermined mode. Generally, eukaryotic cells have a function to refold protein (i.e. modify erroneous modes of intramolecular disulfide bonding into the accurate predetermined mode) in cell organelles such as endoplasmic reticulum. However, prokaryotic cells do not have any such function.

Conventional techniques described above, for example, are not dicrected to basic antimicrobial proteins of such types as described above. They do not disclose or suggest any method for recovering these species of antimicrobial proteins as active types thereof. It is known to modify basic antimicrobial protein recovered in its inactive type into the active type by adding a protein with the chaperon function in vitro. However, this method is so costly that it is hardly used in practice.

In the field of functional protein except for antimicrobial protein, there has been proposed a method for expressing in expression systems of prokaryotes as hosts, a protein of an active type having a predetermined mode of an intramolecular disulfide bond as a fusion protein with a predetermined protein having a refolding function. For example, Japanese Patent Application Laid-open No. 7-250685 proposes a method for expressing a predetermined functional protein in the form of a fusion protein with thioredoxin. Japanese Patent Application Laid-open No. 11-75879 proposes a method for expressing a functional protein in the form of a fusion protein with a protein disulfide isomerase.

Even if an antimicrobial protein of the active type can be expressed by applying these methods to the expression system of the antimicrobial protein, the resulting antimicrobial protein is toxic to the host cell. Therefore, no effective quantity of the antimicrobial protein can be recovered.

### SUMMARY OF THE INVENTION

Accordingly, the following should be satisfied in order to highly efficiently recover basic antimicrobial protein having a predetermined mode of an intramolecular disulfide bond as an active type, in an expressing system of a prokaryote as the host. First, the basic antimicrobial protein should be inactive in the host cell and be expressed without being decomposed in the cell. Second, the basic antimicrobial protein recovered from the host cell should be activated readily by a procedure at low cost. It is an object of the present invention to satisfy the above and provide a method for producing antimicrobial protein.

A first aspect of the invention provides a method for producing antimicrobial protein which comprises: expressing as a fusion protein in a prokaryotic cell, a basic antimicrobial protein A having a predetermined mode of an intramolecular disulfide bond as an active type by combining the protein A with a partner protein B having an isoelectric point below pH 7 and a chaperon function; recovering the fusion protein; and modifying and activating the antimicrobial protein A in the fusion protein into the active type by utilizing a function of the partner protein B.

In accordance with the first aspect, the basic antimicrobial protein A is expressed in the form of a relatively high molecular fusion protein with the partner protein B. Hence, the basic antimicrobial protein A is not decomposed in the host cell. Further, the antimicrobial protein A is generally expressed as an inactive type involving erroneous intramolecular disulfide bond, in prokaryotic host cells. Even if the antimicrobial protein A is expressed as an active type, the toxicity thereof is masked via the fusion with the partner protein B. Thus, the mass expression of antimicrobial protein A can be realized in prokaryotic cells with rapid cell division.

The antimicrobial protein A in the fusion protein recovered from the host cell is refolded by utilizing the function of the partner protein B with the chaperon function during the modification of the antimicrobial protein A into the active type. Thus, the antimicrobial protein A can be activated at low cost. In the activation step, generally, the separation of the antimicrobial protein A from the partner protein B is also carried out. Since the separation is a simple procedure to be selected depending on the fusion state of the fusion protein, the antimicrobial protein A can be activated easily.

In a second aspect of the invention, the fusion protein is expressed by culturing a prokaryotic cell inserted with DNA encoding the fusion protein to express the DNA in the prokaryotic cell.

In the second aspect, representative examples of the method for producing the antimicrobial protein in accordance with the first aspect of the invention are provided.

In a third aspect of the invention, the antimicrobial protein A is any one of thionin, PR protein, lipid transfer protein and ribosome-inactivating protein, all derived from plants, or any one of difensin derived from plants, insects and humans.

The third aspect provides representative examples of the basic antimicrobial protein A having a predetermined mode of an intramolecular disulfide bond as an active type.

In a fourth aspect of the invention, the partner protein B is the following 1 or 2.

1. Protein disulfide isomerase (PDI) or an acid protein encoded by DNA downstream of the nucleotide sequence of thionin derived from plants.

2. Thioredoxin (Tx) or chaperonin.

In such fourth aspect, representative examples of the partner protein B having an isoelectric point below pH 7 and the chaperon function are provided. Among them, PDI or the acid protein encoded by the DNA downstream of thionin from plants is very excellent in terms of the refolding function of the disulfide bond in the antimicrobial protein A.

In a fifth aspect of the invention, the partner protein B comprises an acid partner protein B1 at least with an isoelectric point below pH 7 and a chaperon partner protein B2 at least with a chaperon function.

In such fifth aspect, an advantageous example of the fusion protein is provided. In this case, the fusion protein comprises the following three protein types in fusion, namely the antimicrobial protein A, the acid partner protein B1, and the chaperon partner protein B2.

A sixth aspect of the invention provides a fusion protein comprising a basic antimicrobial protein A having a predetermined mode of an intramolecular disulfide bond as an active type, and a partner protein B having an isoelectric point below pH 7 and a chaperon function.

In such sixth aspect, the antimicrobial protein A and the partner protein B when expressed in host cells can form the fusion protein in a secure manner.

A seventh aspect of the invention provides a fusion protein comprising a basic antimicrobial protein A having a predetermined mode of an intramolecular disulfide bond as an active type; an acid partner protein B1 at least with an isoelectric point below pH 7; and a chaperon partner protein B2 at least with a chaperon function.

In such seventh aspect, examples of the fusion protein are provided, which are different from the examples in the sixth aspect of the invention. In the fusion protein, the acid partner protein B1 and the chaperon partner protein B2 share the functions of the partner protein B as described above.

In an eighth aspect of the invention, the acid partner protein B1 in accordance with the seventh aspect of the invention comprises a carboxyl terminal region of the protein disulfide isomerase from Fumicola insolens, while the chaperon partner protein B2 is peptidylprolyl-cis-trans-isomerase.

In such eighth aspect, there is provided a fusion protein comprising a representative example of the acid partner protein B1 at least with the isoelectric point below pH 7 and a representative example of the chaperon partner protein B2 at least with the chaperon function.

In a ninth aspect of the invention, there is provided a partner protein comprising acid protein B1 at least with an isoelectric point below pH 7 and chaperon partner protein B2 at least with a chaperon function, wherein the partner protein is a protein to be used for the formation of a fusion protein, together with the basic antimicrobial protein A having a predetermined mode of an intramolecular disulfide bond as an active type.

In such ninth aspect, the partner protein to become the partner of the antimicrobial protein A in the fusion protein in the seventh and eighth aspects is provided.

A tenth aspect of the invention provides DNA encoding the fusion protein in the sixth to eighth aspects of the invention. In such tenth aspect, genetic engineering means for producing the fusion protein is provided.

An eleventh aspect of the invention provides a prokaryotic cell where the DNA of the tenth aspect is inserted in an expressible manner. In the eleventh aspect, a specific expression system of the fusion protein is provided, where the DNA of the tenth aspect is utilized.

The above and other advantages of the invention will become more apparent from the following description.

### DETAILED DESCRIPTION OF THE INVENTION

(Fusion Protein)

The fusion protein of the invention is a fusion between the antimicrobial protein A and the partner protein B. Herein, the term “fusion” means a state of two types or more of different protein species in partial or overall integration with a certain force. Examples of the “fusion” include a fusion state of the antimicrobial protein A and the partner protein B chemically bonded together. The fusion state includes the case that the antimicrobial protein A and the partner protein B compose a polypeptide chain in series through a cleavable oligopeptide moiety. The fusion state also includes the state that the antimicrobial protein A and the partner protein B are partially or wholly associated together, through hydrophobic affinity or electric properties or the like. In any of the cases, the antimicrobial protein A is a basic protein where cysteine is present on the amino acid sequence thereof. The partner protein B is at an isoelectric point below pH 7. Hence, the two proteins can be fused together in a secure manner.

(Antimicrobial Protein A)

The antimicrobial protein A of the invention is a basic antimicrobial protein where cysteine is present on the amino acid sequence thereof and which has a predetermined mode of an intramolecular disulfide bond as an antimicrobially active type. The state of the antimicrobial protein A composing the fusion protein of the invention includes a case where the antimicrobial protein A is expressed as an inactive type involving an originally erroneous mode of the disulfide bond therein. The state also includes a case where the antimicrobial protein A is in the steric configuration of the active type due to the formation of the intramolecular disulfide bond of the accurate mode, but the toxicity is masked due to the neutralization of the electric charge or the insolubilization thereof via the fusion with the partner protein B. In another case, because no disulfide bond requisite for the antimicrobially active type can be formed by the fusion with the partner protein B, the antimicrobial protein A is detoxicated.

The type of the antimicrobial protein A is not limited, as long as the antimicrobially active type has a predetermined mode of an intramolecular disulfide bond and it is a basic antimicrobial protein. Preferable examples include any one of thionin, PR protein, lipid transfer protein, and ribosome-inactivating protein, all from plants, or any one of difensin from plants, insects and humans. The thionin from plants is preferably for example thionin from barley, and more specifically thionin comprising amino acids at positions 10 to 54 in the amino acid sequence shown as SEQ ID No: 1.

(Partner Protein B)

The partner protein B of the invention is a protein with an isoelectric point below pH 7 and with the chaperon function. Herein, the term “chaperon” means the function capable of accurately modifying the steric configuration of the subject protein. In accordance with the invention, particularly preferable is the refolding function capable of accurately modifying a wrong bonding position of the intramolecular disulfide bond into a right bonding position in the protein A for the active type. The isoelectric point of the partner protein B is below pH 6, particularly preferably below pH 5.5. The partner protein B may comprise a single partner protein, or the acid partner protein B1 and the chaperon partner protein B2.

The type of the partner protein B is not limited, as long as it satisfies the aforementioned provisions. As the single partner protein B, however, an acid protein (at isoelectric point of pH 3.61) encoded by DNA downstream of the nucleotide sequence of PDI (at isoelectric point of pH 4.68) or thionin derived from plants is preferably exemplified.

Preferred examples of the PDI include PDI derived from Fumicola insolens, specifically PDI comprising amino acids at positions 59 to 543 in the amino acid sequence shown as SEQ ID No: 2. Preferred examples of the acid protein include an acid protein encoded by DNA downstream of the nucleotide sequence of thionin derived from barley, specifically an acid protein comprising amino acids at positions 61 to 124 in the amino acid sequence shown as SEQ ID No: 1.

Further preferable examples of the single partner protein B include Tx (at isoelectric point of pH 5.14), and chaperonins such as GroEL 9 (at isoelectric point of pH 5.08), GroES (at isoelectric point of pH 4.51) and HSP90 (at isoelectric point of pH 4.67).

Examples of the acid partner protein B1 include a carboxyl terminal region of PDI from Fumicola insolens, namely the region (at isoelectric point of pH 3.95) from glutamic acid at the position 514 to leucine at the position 543. Other proteins at isoelectric points in the acidity region may also be used. A preferable example of chaperon partner protein B2 is PPI. The isoelectric point of the chaperon partner protein B2 is not limited. When the isoelectric point is not in the acidity region, however, any means for fusing the chaperon partner protein B2 with the antimicrobial protein A is preferably used for the expression in host cells. Such means includes for example the composition of the antimicrobial protein A and the chaperon partner protein B2 through an oligopeptide moiety enzymatically cleavable in the form of a polypeptide chain in series.

(DNA Encoding Fusion Protein)

According to the inventive method for producing antimicrobial protein, the DNA encoding the fusion protein is inserted in a host prokaryotic cell in an expressible manner to efficiently produce the fusion protein at a mass scale. The nucleotide sequence of such DNA is not specifically limited, as long as the DNA encodes the antimicrobial protein A and the partner protein B.

The coding embodiments include the following. The DNA may encode the antimicrobial protein A and the partner protein B, successively, as a single structural gene. The DNA may encode the antimicrobial protein A, the acid partner protein B1 and the chaperon partner protein B2, successively, as a single structural gene. The DNA may encode the antimicrobial protein A, the intermediate oligopeptide moiety cleavable with appropriate means and the partner protein B, successively, as a single structural gene. The DNA may encode the antimicrobial protein A, the intermediate oligopeptide moiety cleavable with appropriate means, the acid partner protein B1 and the chaperon partner protein B2, successively, as a single structural gene. The antimicrobial protein A and the partner protein B may be encoded as different structural genes. Two or more of the antimicrobial protein A, the acid partner protein B1 and the chaperon partner protein B2 may be encoded as different structural genes. Such DNA includes for example DNA of the nucleotide sequence shown as SEQ ID No: 1 or 2.

(Host Cell with DNA Inserted Therein in Expressible Manner)

The DNA can generally be inserted in an appropriate arbitrary host cell. The type of the host cell is not limited. For example, prokaryotic cells such as Escherichia coli, eukaryotic cells such as yeast, and plant cells and non-human animal cells of any appropriate species may be used. In accordance with the invention, however, prokaryotic cells are used as the host cell.

So as to insert DNA in a host cell in an expressible manner, particularly preferable expression vectors for use include for plasmids such as “pET Expression System” manufactured by Novagen for Escherichia coli. DNA insertion in expression vectors as well as integration of expression vectors into host cells may be carried out by known appropriate methods.

(Method for Producing Antimicrobial Protein)

The method for producing the antimicrobial protein includes a process of producing the fusion protein. This process comprises for example culturing a host cell with the predetermined DNA inserted therein in an expressible manner. The method for culturing the host cell and the conditions for culturing the host cell are not limited.

As the method for recovering the produced fusion protein from the host cell and the purification method thereof, appropriate means may be used as required. Depending on the storage method and storage conditions, the storability of the fusion protein of the invention may sometimes be betterthan that of the antimicrobial protein A. In such case, the method for producing antimicrobial protein may not be completed. For the purpose of the storage and shipping of the fusion protein, instead, only the process of producing and recovering the fusion protein may be carried out. The term “recovery” of the fusion protein may mean the recovery thereof at crude state or at purified state.

The method for producing antimicrobial protein at least comprises modifying the antimicrobial protein A into the antimicrobially active type thereof, utilizing the function of the partner protein B. Generally, the method also comprises a process of separating the antimicrobial protein A from the partner protein B in the fusion protein. The two processes may sometimes progress simultaneously or sequentially over time.

The process of separating the antimicrobial protein A from the partner protein B involves a procedure to cleave the peptide bond in the border of the two proteins, when the two proteins compose a polypeptide chain in series. When an oligopeptide moiety for cleavage is present in the border, a procedure to cleave the oligopeptide moiety is carried out. When the antimicrobial protein A and the partner protein B separately comprise individual polypeptide chains and these proteins are associated together via electric properties and the like, a process of separating these proteins is carried out.

The process of modifying the antimicrobial protein A into the antimicrobially active type thereof may progress and be completed automatically, owing to the function of the partner protein B. However, other appropriate procedures for refolding disulfide bond may be concurrently carried out, satisfactorily, for the promotion of the process.

### EMBODIMENTS

### Example 1

(Construction of Gene of Fusion of Thionin-Acid Protein)

There was prepared a gene encoding a fusion of thionin derived from barley and an acid protein encoded by the DNA downstream of the nucleotide sequence of thionin from barley. The gene was cloned in a site scissored out via the cleavage with restriction endonucleases NdeI and BamHI in the plasmid pET-19b manufactured by Novagen.

The thionin gene from barley is of the nucleotide sequence at position 28 to position 162 in SEQ ID No: 1. The gene of the acid protein is of the nucleotide sequence at position 181 to position 372 in SEQ ID No: 1. In SEQ ID No: 1, the nucleotide sequence CATATG at position 7 to position 12 corresponds to a cleavage region with restriction endonuclease NdeI, while the nucleotide sequence GGATCC at position 378 to 383 corresponds to a cleavage region with restriction endonuclease BamHI. In the amino acid sequence concurrently depicted, the amino acid sequence Thr-Glu-Gly-Arg at position 5 to 8 and position 56 to 59 corresponds to a recognition site of Factor Xa as protease.

### Example 2

(Construction of Gene of Fusion of Thionin and PDI)

There was prepared a gene encoding a fusion protein of thionin derived from barley and PDI from Fumicola insolens. The gene was inserted in a site scissored out via the cleavage with restriction endonucleases NdeI and BamHI in the plasmid pET-19b manufactured by Novagen for cloning.

The thionin gene from barley is of the nucleotide sequence at position 25 to position 159 in SEQ ID No: 2. The PDI gene is of the nucleotide sequence at position 175 to position 1629 in SEQ ID No: 2. In SEQ ID No: 2, the nucleotide sequence CATATG at position 7 to position 12 corresponds to a cleavage region with restriction endonuclease NdeI, while the nucleotide sequence GGATCC at position 1635 to 1640 corresponds to a cleavage region with restriction endonuclease BamHI. In the amino acid sequence concurrently depicted, the amino acid sequence Thr-Glu-Gly-Arg at position 5 to 8 and position 55 to 58 corresponds to a recognition site of Factor Xa.

### Comparative Example 1

(Construction of Thionin Gene)

The gene encoding thionin derived from barley was inserted in a site scissored out via the cleavage with restriction endonucleases NdeI and BamHI in the plasmid pET-19b manufactured by Novagen for cloning. The thionin gene from barley is of the nucleotide sequence at position 28 to position 162 in SEQ ID No: 3. In SEQ ID No: 3, the nucleotide sequence CATATG at position 7 to position 12 corresponds to a cleavage region with restriction endonuclease NdeI, while the nucleotide sequence GGATCC at position 168 to 173 corresponds to a cleavage region with restriction endonuclease BamHI. In the amino acid sequence concurrently depicted, alternatively, the amino acid sequence Thr-Glu-Gly-Arg at position 5 to 8 corresponds to a recognition site of Factor Xa.

### Comparative Example 2

(Construction of Gene of Fusion of Thionin and PDI Acid Region)

At the carboxyl terminus of PDI from Fumicola insolens, there is an acid region abundant in acid amino acids. The gene encoding a fusion protein of thionin from barley and the acid region was prepared. The gene was inserted in a site scissored out via the cleavage with restriction endonucleases NdeI and BamHI in the plasmid pET-19b manufactured by Novagen for cloning.

Because the acid region never contains the active site of PDI, it is deemed that the acid region does not have a catalytic action to refold disulfide bond.

The thionin gene from barley is of the nucleotide sequence at position 25 to position 159 in SEQ ID No: 4. The gene of the acid region is of the nucleotide sequence at position 175 to position 264. In SEQ ID No: 4, the nucleotide sequence CATATG at position 7 to position 12 corresponds to a cleavage region with restriction endonuclease NdeI, while the nucleotide sequence GGATCC at position 270 to 275 corresponds to a cleavage region with restriction endonuclease BamHI. In the amino acid sequence concurrently depicted, the amino acid sequence Thr-Glu-Gly-Arg at position 5 to 8 and position 35 to 38 corresponds to a recognition site of Factor Xa.

[Gene Expression in Escherichia coli]

With the vectors constructed in Examples 1 and 2 and Comparative Examples 1 and 2, Escherichia coli BL21 (DE3) pLysS was transformed. The individually recovered transformants were overnight cultured in the LB culture medium (1% bacto-tryptone, 0.5% bacto-yeast extract, 1% NaCl) and were inoculated at 1% in the LB culture medium. Then, the transformants were cultured at 37° C. until OD=0.5, to which was added IPTG (isopropylthio-β-D-galatoside) to a final concentration of 1 mM, for expression induction for 6 hours.

Subsequently, the culture broth was centrifuged to harvest the cells, followed by addition of a sonication buffer at a weight of 10-fold those of the wet cells, for suspension. After the cells were disrupted via ultrasonication, the cells were centrifuged at 15,000 rpm for 30 minutes. The supernatant was designated soluble fraction, while the precipitate was designated insoluble fraction.

These were subjected to SDS-PAGE. Consequently, the thionin was not expressed in Comparative Example 1. However, it was observed that the fusion proteins of the Examples and the remaining Comparative Example were all expressed in the insoluble fractions.

[Recovery of Fusion Protein]

The insoluble fractions of the fusion proteins of the Examples 1 and 2 and the Comparative Example 2 and the insoluble fraction from the culture of Escherichia coli BL21 (DE3) pLysS with no inserted vector therein as the control for the assay of antimicrobial activity were rinsed twice in 0.5% Triton X-100/1 mM EDTA. Subsequently, a urea solution (8 M urea, 50 mM Tris-HCl at pH 8.0, 1 mM DTT, 1 mM EDTA) was added for solubilization. After centrifugation, the supernatant was transferred to a dialysis tube, for one-hour dialysis at 4° C. against 4M urea solution. During the dialysis against the urea solution containing DTT, the disulfide bond in thionin may be cleaved.

Subsequently, the outer dialysis solution was sequentially exchanged to 2M urea solution and the buffer (50 mM Tris-HCl at pH 8.0, 100 mM NaCl, 1 mM calcium chloride) for protease cleavage. Thereafter, dialysis was carried out overnight against the latter buffer. During the dialysis, the disulfide bond of thionin may be refolded. After the termination of the dialysis and centrifugation, the resulting supernatant was used for the cleavage with Factor Xa as described below.

During the procedures, the refolding efficiency with PDI or the acid protein can be expected to be improved in the fusion protein between thionin and PDI or the fusion protein between thionin and the acid protein (Example 1 or 2), in addition to the refolding effect of thionin due to the dialysis per se.

[Cleavage of Fusion Protein with Protease]

As shown in SEQ ID No: 1, 2 or 4, the fusion proteins of Examples 1 and 2 and Comparative Example 2 thus dialyzed retained the recognition sequence of a protease Factor Xa in between thionin and the partner protein. Then, the fusion protein was subjected to cleavage with Factor Xa of 30 μg per 1 mg of each fusion protein described above at 30° C. overnight.

[Assay of Antimicrobial Activity]

The protein solution recovered by the cleavage procedures was concentrated to 30 μl using Microcon-3 (MWCO: 3000) as a concentration unit with a filtration membrane. Such great number of concentrated sample solutions were divided at 30 μl each in the individual wells of a 96-well microplate, followed by addition of 10 μl of 200 mM MES [2-(N-morpholino)ethanesulfonic acid] buffer at pH 6 and 60 μL of a spore suspension of Ceratocystis fimbriata (100,000 spores per 1 ml-80% potato dextrose broth). Then, the resulting mixtures were cultured at 26° C. On the other hand, the insoluble fraction of the culture of Escherichia coli with none of the vectors inserted therein was treated in the same manner as the control.

Then, the absorbance 30 minutes and 48 hours later was measured at 415 nm with a microplate reader of Model 3550. The growth inhibition was determined by the following formula (1). Herein, A represents the value obtained by subtracting the absorbance measured 30 minutes later from that measured 48 hours later in each concentrated sample solution, and B represents the same value in a solution not containing the sample.

Growth inhibition (%)=(B−A)×100/B (1)

Consequently, the growth inhibition of the control was at 9.6%. The growth inhibition in Comparative Example 2 was at 11.5%, with no substantial antimicrobial activity observed. The growth inhibition in Example 1 was at 99.8%, while the growth inhibition in Example 2 was at 99.1%. In both the Examples, high antimicrobial activity was observed. The difference between Comparative Example 2 and Examples 1 and 2 as described above may possibly be due to the presence or absence of the effect on the improvement of the refolding efficiency owing to the partner protein.

While the preferred embodiment has been described, variations thereto will occur to those skilled in the art within the scope of the present inventive concepts which are delineated by the following claims.

What is claimed is:

1. A method for producing antimicrobial protein, comprising expressing as a fusion protein in a prokaryotic cell, a basic antimicrobial protein A having a predetermined mode of an intramolecular disulfide bond as an active type by combining the protein A with a partner protein B having an isoelectric point below pH 7 and a chaperon function,
recovering the fusion protein, and
modifying and activating the antimicrobial protein A in the fusion protein into the active type by utilizing a function of the partner protein B.

2. The method according to claim 1, wherein the fusion protein is expressed by culturing a prokaryotic cell inserted with DNA encoding the fusion protein to express the DNA in the prokaryotic cell.

3. The method according to claim 1, wherein the antimicrobial protein A is any one of thionin, PR protein, lipid transfer protein and ribosome-inactivated protein, all derived from plants, or any one of difensin derived from plants, insects and humans.

4. The method according to claim 1, wherein the partner protein B comprises an acid partner protein B1 at least with an isoelectric point below pH 7 and a chaperon partner protein B2 at least with a chaperon function.

5. The method according to claim 1, further comprising separating the antimicrobial protein A from the partner protein B in the fusion protein and modifying the antimicrobial protein A into an antimicrobially active type by utilizing the function of the partner protein B.

6. The method according to claim 5, wherein the step of separating the antimicrobial protein A from the partner protein B includes the cleavage of a peptide bond in the border of the two proteins.

7. The method according to claim 5, wherein the step of separating the antimicrobial protein A from the partner protein B includes the cleavage of an oligopeptide moiety interposed in the border of the two proteins for cleavage.

8. The method according to claim 5, wherein the step of modifying the antimicrobial protein A into the antimicrobially active type thereof includes a general refolding procedure of disulfide bond for promoting the modification.

9. A fusion protein comprising
a basic antimicrobial protein A having a predetermined mode of an intramolecular disulfide bond as an active type, and
a partner protein B having an isoelectric point below pH 7 and a chaperon function.

10. The fusion protein according to claim 9, wherein the antimicrobial protein A and the partner protein B are chemically bonded together.

11. The fusion protein according to claim 9, wherein the antimicrobial protein A and the partner protein B form in series a polypeptide chain through an oligopeptide moiety enzymatically cleavable.

12. The fusion protein according to claim 9, wherein the antimicrobial protein A and the partner protein B are partially or wholly associated together via hydrophobic affinity or electric properties.

13. The fusion protein according to claim 9, wherein the antimicrobial protein A is any one of thionin, PR protein, lipid transfer protein and ribosome-inactivating protein, all derived from plants, or any one of difensin derived from plants, insects and humans.

14. The fusion protein according to claim 9, wherein the partner protein B is thioredoxin (Tx) or chaperonin.

15. The fusion protein according to claim 9, wherein the chaperon function of the partner protein B is a refolding function to modify a wrong bonding position of the intramolecular disulfide bond into a right bonding position in the protein A for the active type thereof.

16. The fusion protein according to claim 9, wherein the partner protein B is protein disulfide isomerase (PDI) or an acid protein encoded by DNA downstream of the nucleotide sequence of thionin derived from plants.

17. A fusion protein comprising: a basic antimicrobial protein A having a predetermined mode of an intramolecular disulfide bond as an active type; an acid partner protein B1 at least with an isoelectric point below pH 7; and a chaperon partner protein B2 at least with a chaperon function.

18. The fusion protein according to claim 17, wherein the acid partner protein B1 comprises a carboxyl terminal region of the PDI derived from Fumicola insolens and the chaperon partner protein B2 is peptidylprolyl-cis-trans-isomerase.

19. A partner protein comprising an acid partner protein B1 at least with an isoelectric point below pH 7 and a chaperon partner protein B2 at least with a chaperon function, wherein the partner protein is a protein to be used for the formation of a fusion protein, together with a basic antimicrobial protein A having a predetermined mode of an intramolecular disulfide bond as an active type.

20. DNA encoding a fusion protein according to claim 1.
